(19) United States
(12) Patent Application Publication
(10) Pub. No.: US 2023/0285187 A1
(43) Pub. Date: Sep. 14, 2023

(54) ENDORECTAL COOLING DEVICE HOLDER

(71) Applicant: The Board of Regents of The University of Texas System, Austin, TX (US)

(72) Inventor: Xiaosong MENG, Dallas, TX (US)

(21) Appl. No.: 18/177,265

(22) Filed: Mar. 2, 2023

Related U.S. Application Data
(60) Provisional application No. 63/318,533, filed on Mar. 10, 2022.

(51) Int. Cl. A61F 7/12

(52) U.S. Cl. CPC A61F 7/12

(57) ABSTRACT

An endorectal cooling device holder is provided. The holder includes a base operable to be coupled to a surface, an arm extending from the base, and a receiver pivotably coupled with the arm. The receiver forms a receiving aperture operable to receive an endorectal cooling device and maintain the position of the endorectal cooling device.

### CROSS-REFERENCE TO RELATED APPLICATION

This application claims the benefit of U.S. Provisional Patent Application Ser. No. 63/318,533 filed on Mar. 10, 2022, the content of which is incorporated by reference in its entirety.

### BACKGROUND

### 1. Field

The present disclosure relates generally to systems and methods related to endorectal cooling devices. In at least one example, the present disclosure relates to a holder operable to maintain the positioning of a endorectal cooling device.

### 2. Discussion of Related Art

Endorectal cooling devices are used during Transurethral Ultrasound Ablation (TULSA) procedures for treatment of prostate cancer. TULSA is a minimally invasive procedure that uses directional ultrasound to produce very high temperatures to ablate (destroy) targeted prostate tissue. An ultrasound applicator is inserted into the urethra, and the endorectal cooling device is inserted into the rectum. As the ultrasound applicator emits ultrasound energy to the prostate, the endorectal cooling device protects the rectal tissue from the thermal ultrasound energy, helping to preserve the patient's natural functions and prevent complications of treatment such as rectal injury or rectourethral fistulas. The endorectal cooling device must maintain its position and orientation to function as desired.

### BRIEF SUMMARY

The present inventive concept provides for a holder operable to receive and maintain the position and orientation of an endorectal cooling device during a TULSA procedure.

The aforementioned may be achieved in an aspect of the present inventive concept by providing a holder for an endorectal cooling device. The holder may include a base operable to be coupled to a surface, an arm extending from the base, and a receiver pivotably coupled with the arm. The receiver may form a receiving aperture operable to receive an endorectal cooling device and maintain the position of the endorectal cooling device.

The receiver aperture may be substantially rectangular. The receiving aperture may have a width between about 39 millimeters and 42 millimeters and a height between about 3 millimeters and about 36 millimeters. The receiving aperture may form a notch operable to receive a ridge on the endorectal cooling device. The base may include an adhesive which permits the base to be removably coupled to the surface. The adhesive may include a micro-suction tape. The base may include a top plate and a bottom plate. The bottom plate may be operable to be received in a recess formed in a bottom surface of a system base for a positioning system. The top plate may be operable to be positioned on a top surface of the system base opposite the bottom surface. The bottom plate may include a fastener that is operable to be received by the top plate. The fastener may be operable to couple the top plate with the bottom plate. The fastener may be operable to be inserted through a channel in the system base and through an aperture formed in the top plate. The fastener may include a screw. The base may include a nut operable to be threadedly coupled with the screw extending through the top plate to couple the top plate with the bottom plate. The arm may be rotatable in relation to the base. The arm may be translatable along a longitudinal axis of the base. The arm may be coupled with the base with a pin. The receiver may be pivotable in relation to the arm. The arm may include a bracket operable to receive the receiver. The arm may include two longitudinal beams extending along a longitudinal axis of the arm and at least one cross-beam connecting the two longitudinal beams.

The aforementioned may also be achieved in an aspect of the present inventive concept by providing a system including an endorectal cooling device and a holder operable to receive and maintain the position of the endorectal cooling device. The holder may include a base operable to be coupled to a surface, an arm extending from the base, and a receiver pivotably coupled with the arm. The receiver may form a receiving aperture operable to receive an endorectal cooling device and maintain the position of the endorectal cooling device.

The aforementioned may also be achieved in an aspect of the present inventive concept by providing a kit including a holder for an endorectal cooling device and a syringe holder operable to receive a syringe. The holder may include a base operable to be coupled to a surface, an arm extending from the base, and a receiver pivotably coupled with the arm. The receiver may form a receiving aperture operable to receive an endorectal cooling device and maintain the position of the endorectal cooling device.

The foregoing is intended to be illustrative and is not meant in a limiting sense. Many features of the embodiments may be employed with or without reference to other features of any of the embodiments. Additional aspects, advantages, and/or utilities of the present inventive concept will be set forth in part in the description that follows and, in part, will be apparent from the description, or may be learned by practice of the present inventive concept.

### BRIEF DESCRIPTION OF THE DRAWINGS

The description will be more fully understood with reference to the following figures and data graphs, which are presented as various embodiments of the present inventive concept and should not be construed as a complete recitation of the scope of the present inventive concept, wherein:

FIG. 1A illustrates an example of a system for a transurethral ultrasound ablation (TULSA) procedure which may be used in accordance with the present disclosure;

FIG. 1B illustrates an enlarged view of a portion of the system of FIG. 1A;

FIG. 10 illustrates another perspective view of a portion of the system of FIG. 1A;

FIG. 2A illustrates a rear perspective view of a holder for an endorectal cooling device;

FIG. 2B illustrates a rear view of the holder of FIG. 2A;

FIG. 2C illustrates an exploded view of the holder of FIG. 2A;

FIG. 3A illustrates a receiver operable to receive the endorectal cooling device;

FIG. 3B illustrates the receiver receiving the endorectal cooling device;

FIG. 4A illustrates a holder with a hydraulic base;

FIG. 4B illustrates the holder of FIG. 4A, where the arm is translated along a longitudinal axis of the base;

FIG. 5A illustrates a holder with a rack base;

FIG. 5B illustrates a front perspective view of the holder of FIG. 5A;

FIG. 5C illustrates an enlarged view of the base of the holder of FIG. 5A;

FIG. 5D illustrates the holder of FIG. 5A with a wedge preventing rearward movement along the base

FIG. 6A illustrates a holder with a base operable to be retrofittedly coupled with a system base;

FIG. 6B illustrates an exploded view of the base of FIG. 6A;

FIG. 7A illustrates the holder of FIG. 6A coupled with a system base;

FIG. 7B illustrates a bottom view of the system base coupled with the holder of FIG. 6A; and

FIG. 7C illustrates an enlarged top, perspective view of FIG. 7B, with the holder omitting the arm, the bracket, and the receiver.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims. Further, it should be understood that any one of the features of the present inventive concept may be used separately or in combination with other features. Other systems, methods, features, and advantages of the present inventive concept will be, or become, apparent to one with skill in the art upon examination of the figures and the detailed description. It is intended that all such additional systems, methods, features, and advantages be included within this description, be within the scope of the present inventive concept, and be protected by the accompanying claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

II. General Architecture

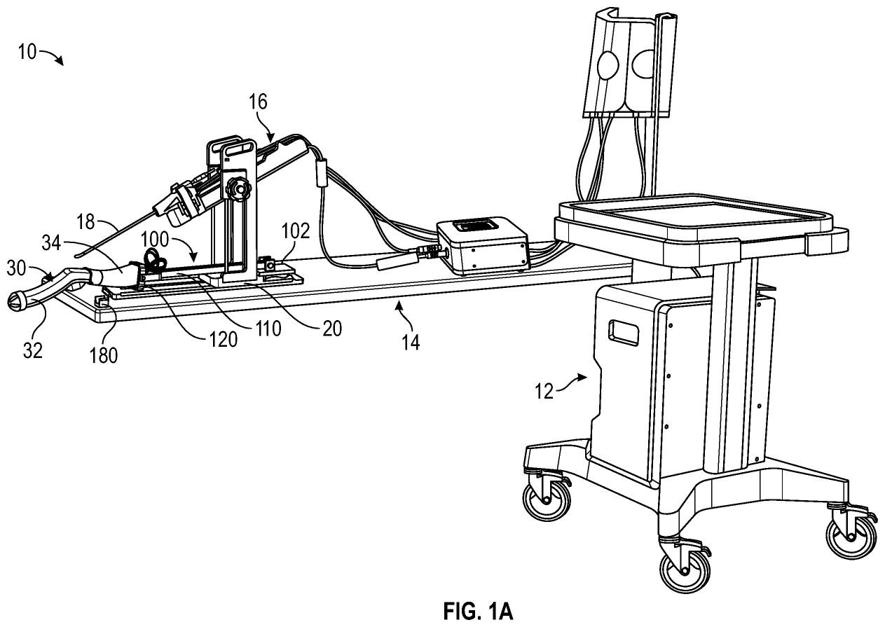

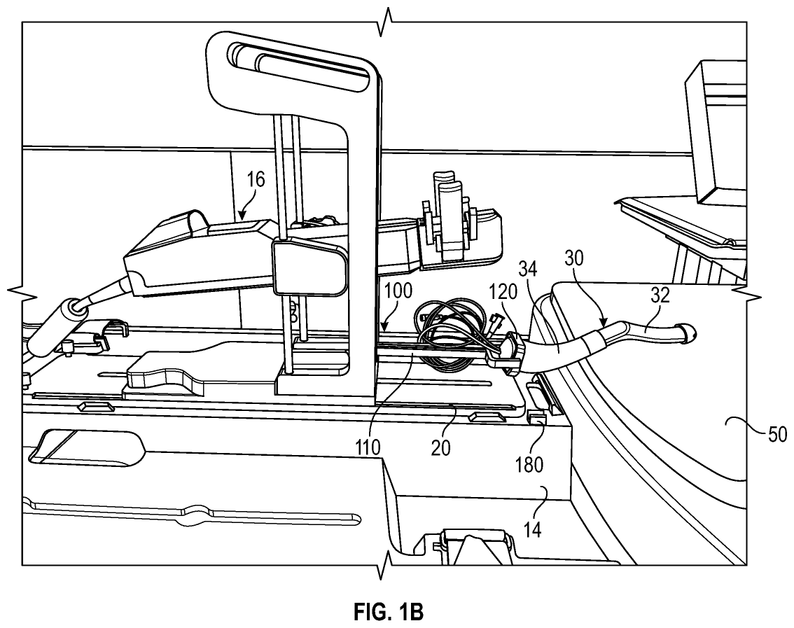

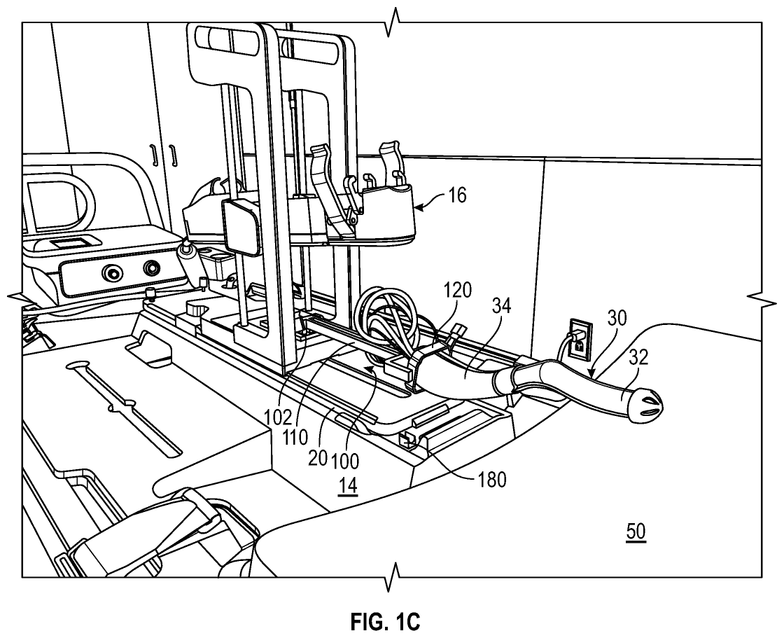

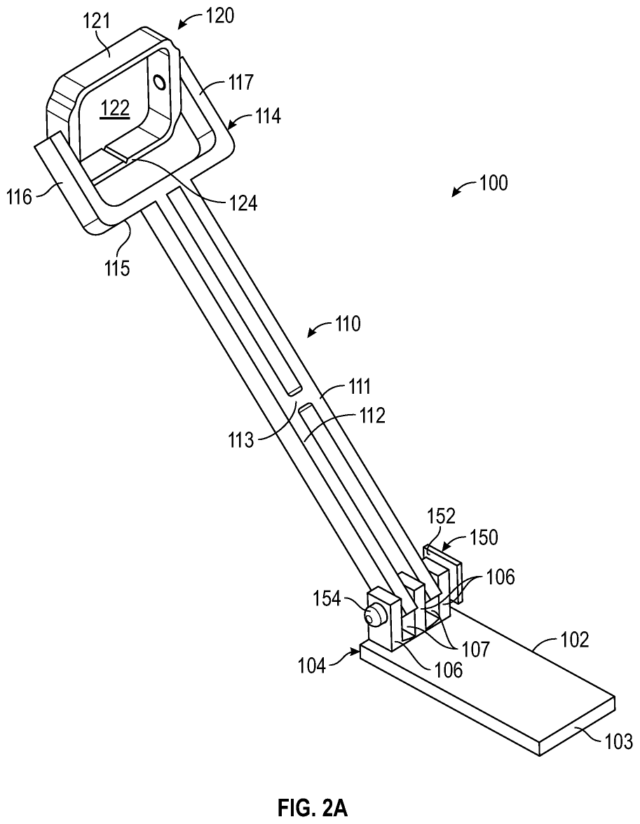

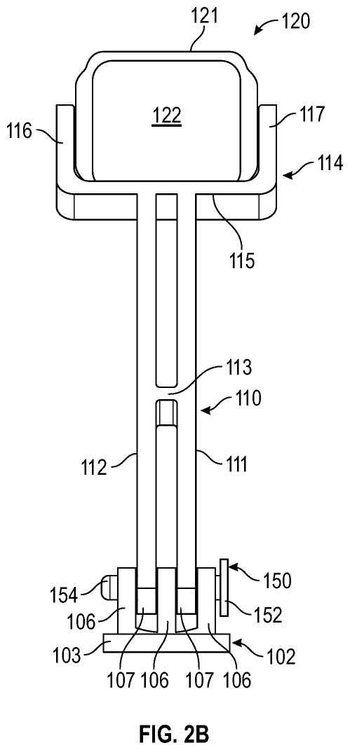

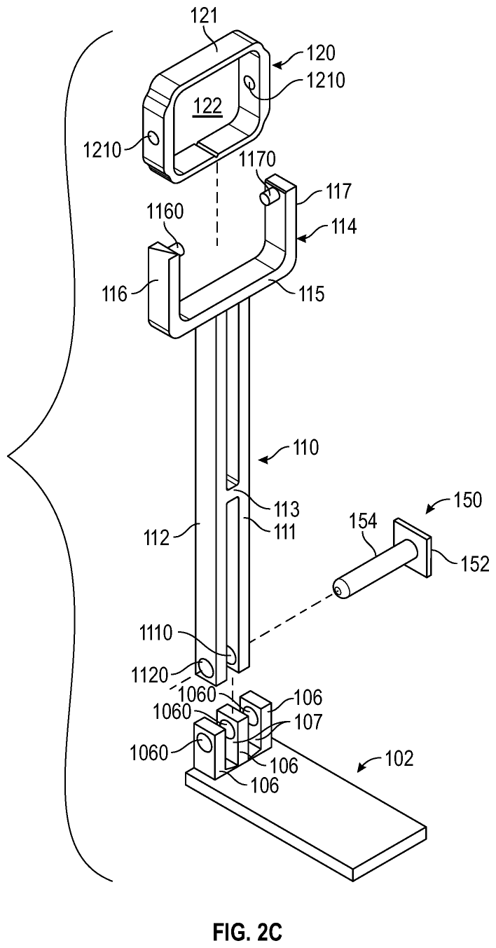

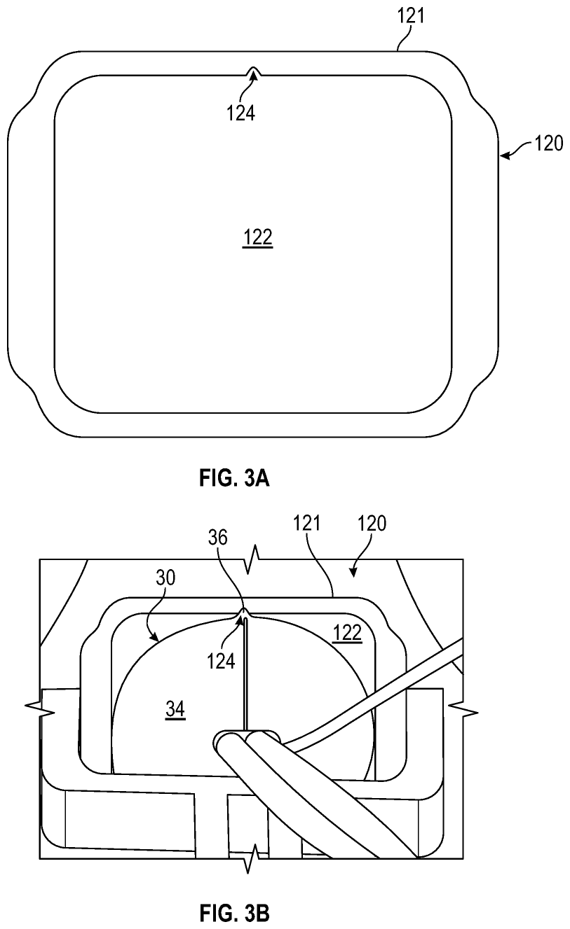

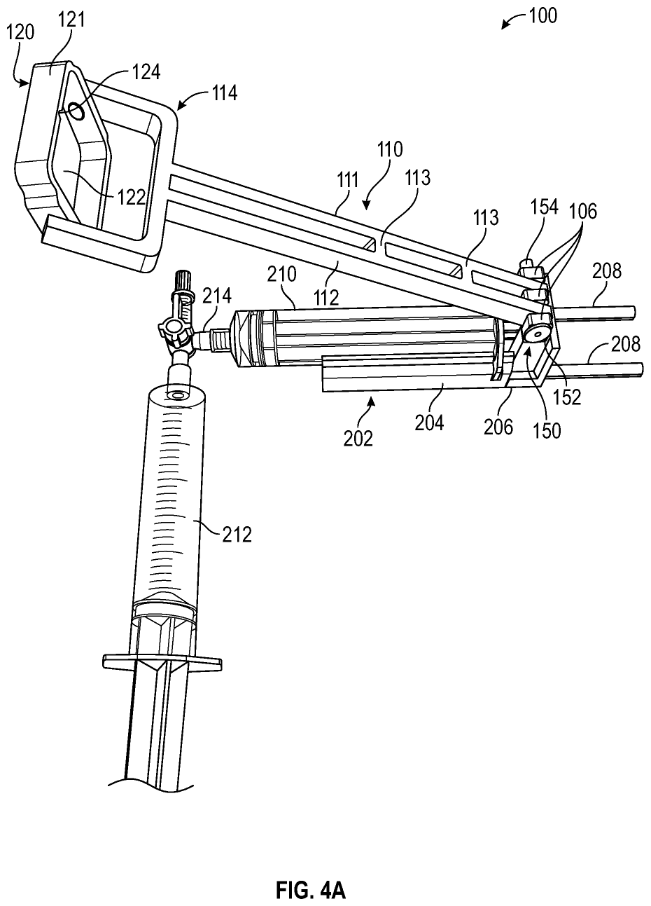

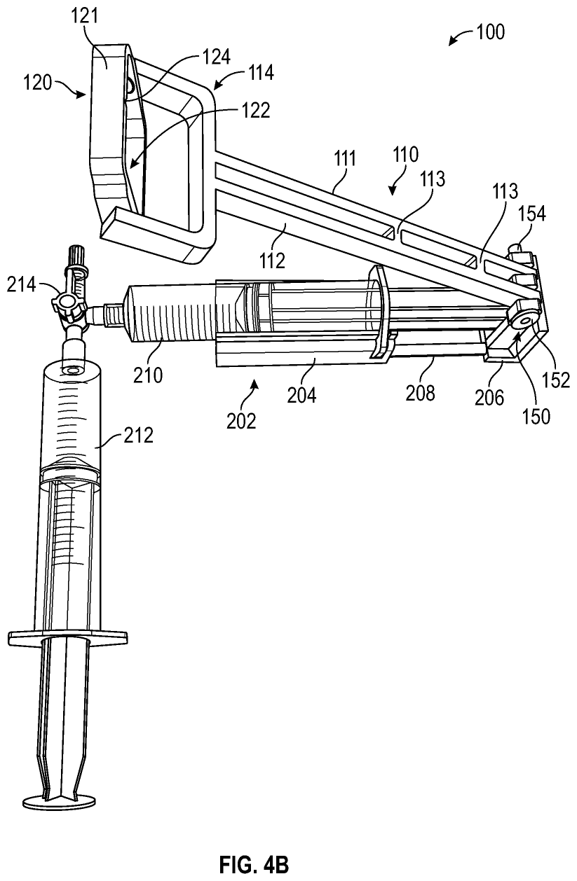

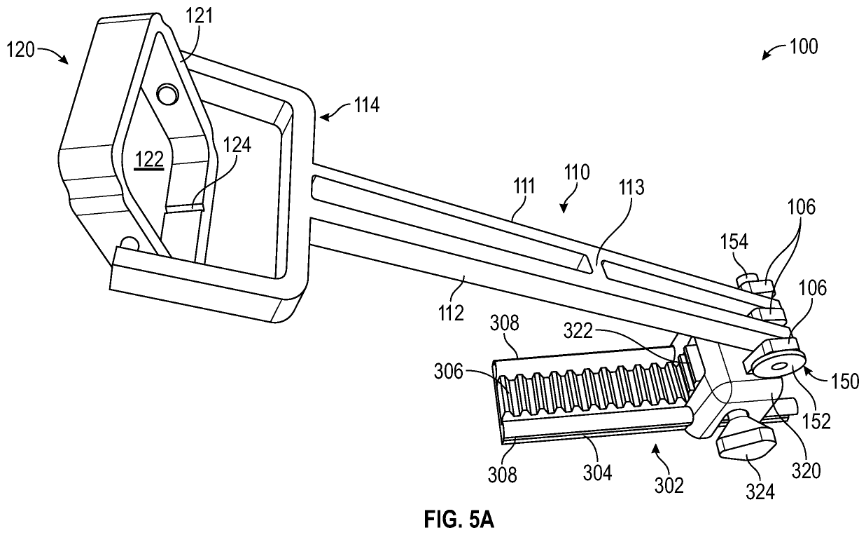

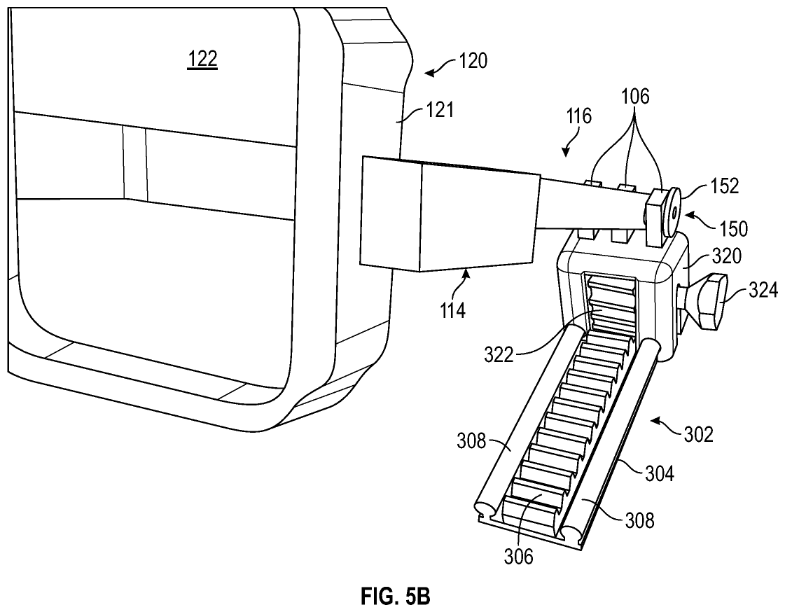

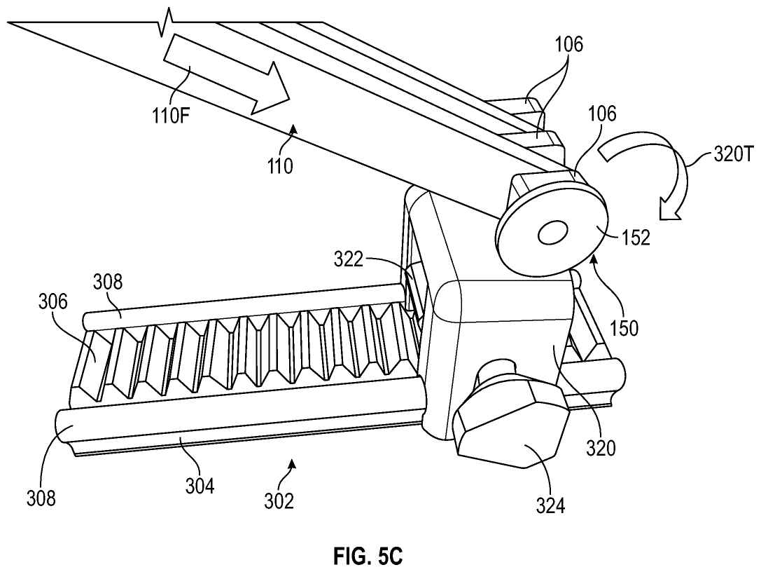

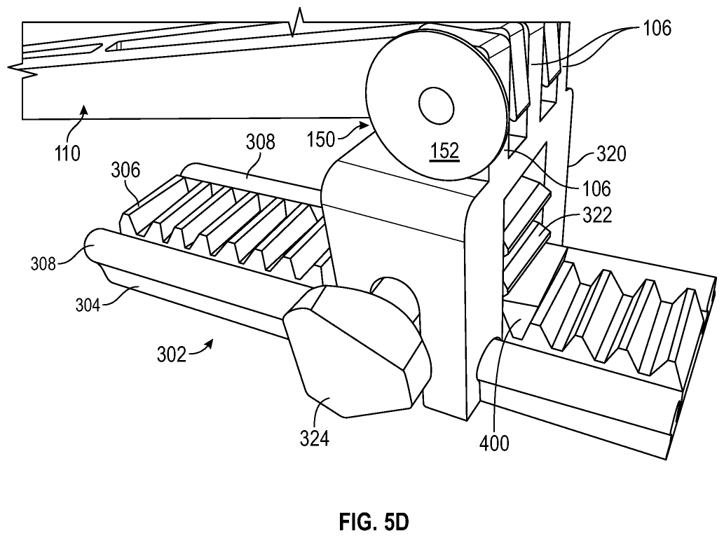

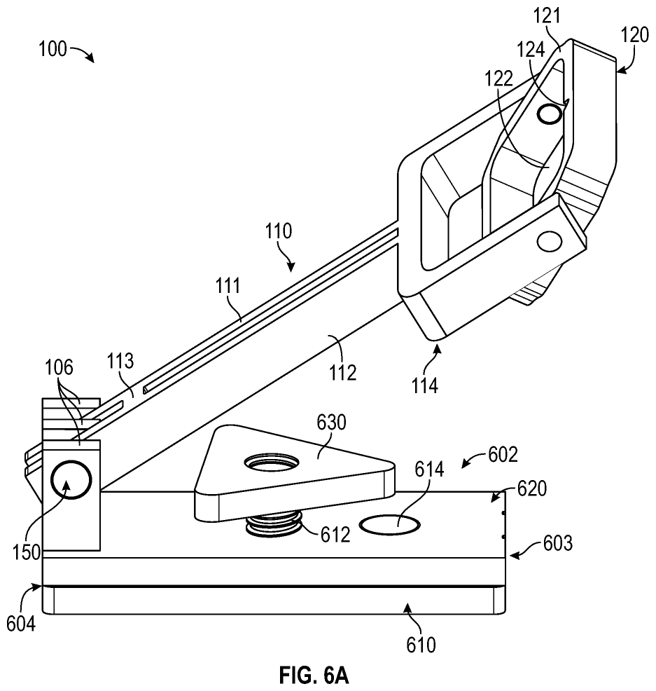

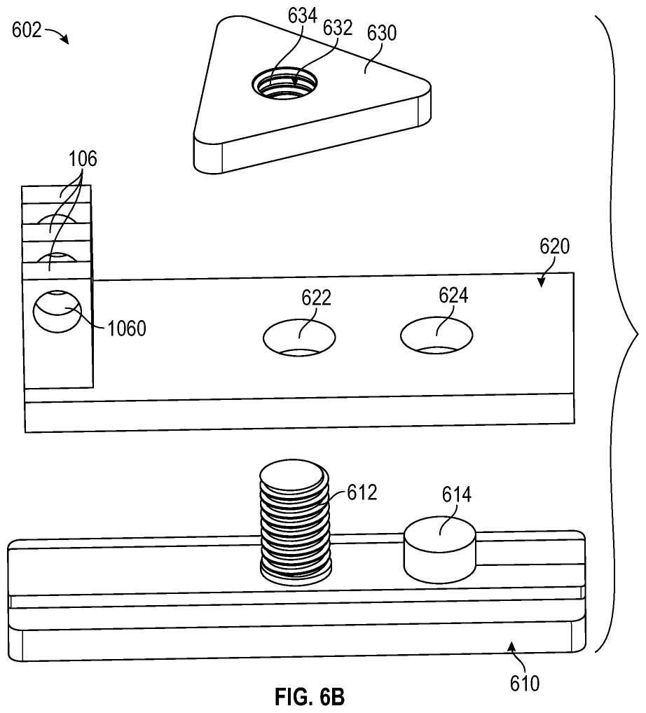

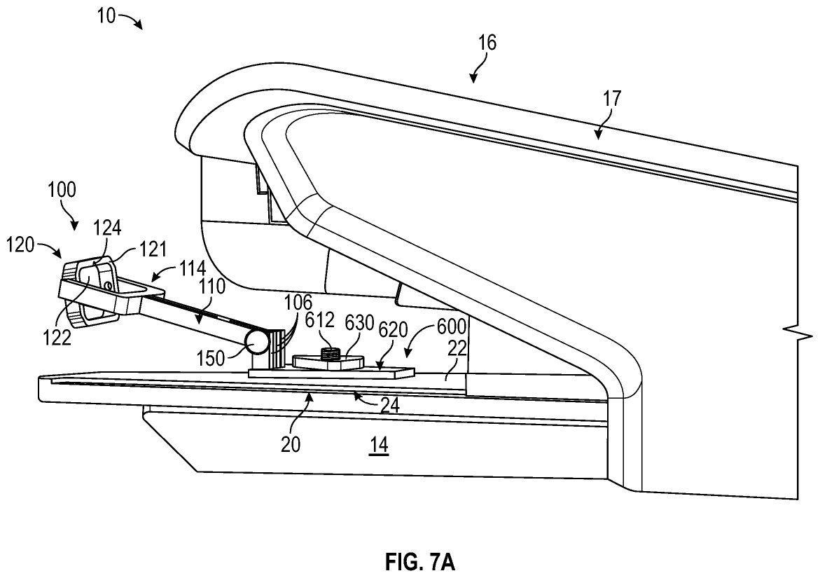

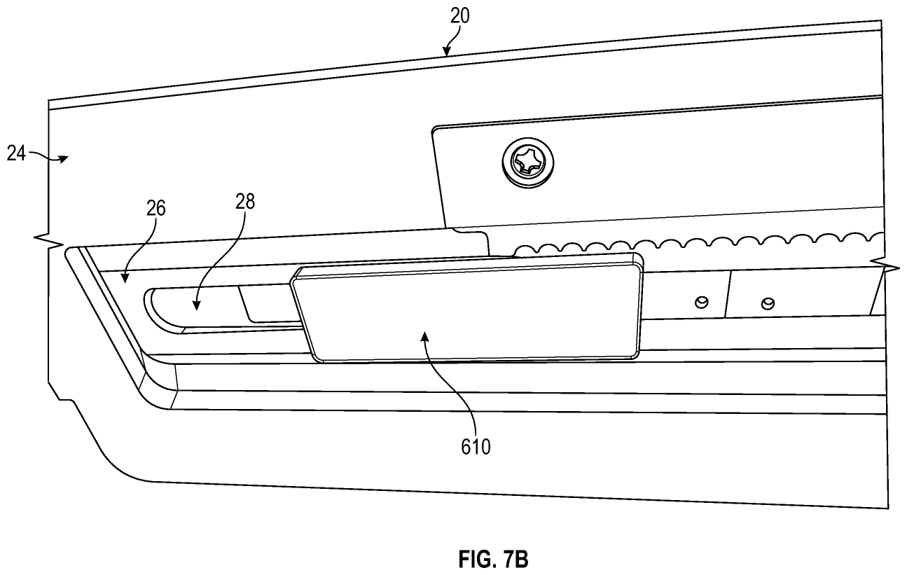

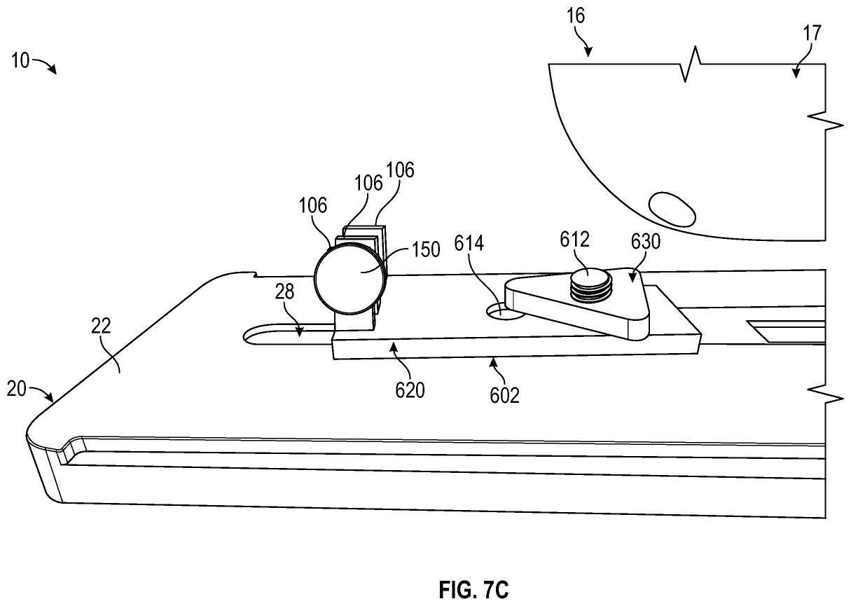

The disclosure now turns to FIGS. 1A, 1B, and 1C, which illustrate an exemplary environment or system 10 for a holder 100 for an endorectal cooling device 30, in which the present disclosure may be implemented. The system 10 can include an endorectal cooling device 30 and a holder 100 operable to receive and maintain the position of the endorectal cooling device 30. The endorectal cooling device 30 can be used during Transurethral Ultrasound Ablation (TULSA) procedures for treatment of prostate cancer. TULSA is a minimally invasive procedure that uses directional ultrasound to produce very high temperatures to ablate (destroy) targeted prostate tissue. An ultrasound applicator 18 is operable to be inserted into the urethra, and the endorectal cooling device 30 is operable to be inserted into the rectum. As the ultrasound applicator 18 emits ultrasound energy to the prostate, the endorectal cooling device 30 protects the rectal tissue from the thermal ultrasound energy, helping to preserve the patient's natural functions and prevent complications.

The ultrasound applicator 18 is positioned and oriented by the positioning system 16. The ultrasound applicator 18 can extend from and/or be coupled with the positioning system 16. System electronics 12 are operable to control the function and/or receive data from the ultrasound applicator 18 and/or the endorectal cooling device 30. The system 10 can be disposed on a surface 14, such as a table or a tray. In at least one example, the positioning system 16 can include a system base 20. In some examples, the system base 20 can be operable to be set on the surface 14. In some examples, the system base 20 can also be considered the surface 14.

In at least one example, the system 10 can include a syringe holder 180 which is operable to hold a syringe (not shown). For example, the syringe can be a 30 milliliter syringe. The syringe can be used during the TULSA procedure to inflate and/or deflate an endorectal balloon (not shown). In at least one example, the syringe holder 180 can be provided along with the holder 100 as a kit for TULSA procedures. The syringe holder 180 can have a recess that is operable to receive the syringe. The recess can have a cylindrical shape. In some examples, the recess can have a diameter between about 20 millimeters and about 30 millimeters. Alternately, the recess can have a diameter between about 23 millimeters and about 27 millimeters, alternately between about 24 millimeters and about 25 millimeters, alternately about 24.5 millimeters. In other examples, the syringe holder 180 can have any size and shape suitable to receive the syringe, for example via friction fit. In some examples, the syringe holder 180 can be coupled to the surface 14, 20 to maintain the position of the syringe holder 180. As illustrated in FIGS. 1A-1C, the syringe holder 180 is disposed on the surface 14. In other examples, the syringe holder 180 can be disposed on the base 20 functioning as the surface. For example, the syringe holder 180 can be coupled to the surface 14, 20 by adhesion, by microsuction, or by any other suitable mechanism. By maintaining the position of the syringe holder 180 on the surface 14, 20, the syringe holder 180 can be easily accessible and does not move as the syringe is placed in and/or on the syringe holder 180.

The endorectal cooling device 30 includes an insertion portion 32 and a handle portion 34. The insertion portion 32 is operable to be inserted into a patient's rectum. The handle portion 34 remains outside of the patient's body such that the user can hold and/or move the endorectal cooling device 30. As the ultrasound applicator 18 emits ultrasound energy to the prostate, the endorectal cooling device 30 protects the rectal tissue from the thermal ultrasound energy by lowering the temperature of the rectal tissue. The endorectal cooling device 30 must cool the corresponding tissue that is being heated by the ultrasound applicator 18, or the endorectal cooling device 30 is not achieving its purpose and the tissue can be damaged. Accordingly, once positioned in the patient, the endorectal cooling device 30 must maintain its position and orientation to function as desired. However, endorectal cooling device 30 may move in the patient's rectum which can affect the positioning and orientation of the endorectal cooling device 30.

As illustrated in FIGS. 1A-1C, the holder 100 is operable to receive and maintain the position of the endorectal cooling device 30. Conventionally, the endorectal cooling device 30 is held in place with either tape to the patient's buttocks or with a sandbag. However, the conventional methods do not securely hold the endorectal cooling device 30 in place, allowing the endorectal cooling device 30 to either pull out or rotate, which can impact the performance of the procedure. The holder 100 disclosed herein is operable to securely hold the endorectal cooling device 30 substantially parallel to the MRI table so that the endorectal cooling device 30 does not rotate and does not move. In at least one example, as shown in FIGS. 1A-1C, the holder 100 is positioned on the system base or surface 20. In some examples, the holder 100 is operable to be positioned on the surface 14 as the table or tray or any other suitable surface 14, 20.

Referring to FIGS. 1A-2C, the holder 100 includes a base operable to be coupled to the surface 14, 20, an arm 110 extending from the base 102, and a receiver 120 coupled with the arm 110.

In at least one example, the base 102 of the holder 100 is operable to be coupled to the surface 14, 20. In some examples, the base 102 can include an adhesive which permits the base 102 to be removably coupled to the surface 14, 20. For example, the adhesive can include a microsuction tape. In some examples, the base 102 can include a suction cup operable to removably couple the base 102 to the surface 14, 20. In at least one example, when coupled with the surface 14, 20, the base 102 is operable to be lifted vertically from the surface 14, 20 to be removed. The base 102 may resist and/or prevent horizontal movement such as sliding to ensure the positioning of the holder 100 is maintained. In at least one example, the base 102 can have a length between about 70 millimeters and about 90 millimeters, alternately between about 75 millimeters and about 85 millimeters, alternately about 80 millimeters. The width of the base 102 can be between about 25 millimeters and about 45 millimeters, alternately between about 30 millimeters and about 40 millimeters, alternately about 35 millimeters. The size of the base 102 is configured to provide sufficient surface area for coupling with the surface 14, 20. For example, with microsuction tape, the base 102 needs to have sufficient size so that the microsuction tape can hold on to the surface 14, 20 without lifting off the surface during the procedure. Accordingly, the base 102 must be sufficiently coupled with the surface 14, 20 to prevent undesired movement of the holder 100 and consequently the endorectal cooling device 30.

The patient's body may push against the endorectal cooling device 30 to eject the endorectal cooling device 30 from the rectum, so the holder 100 must resist that force to prevent the endorectal cooling device 30 from being pulled/pushed out of the rectum and/or rotate, which can impact the performance of the procedure. The base 102, as illustrated herein, is substantially rectangular in shape, as much of the force enacted upon the endorectal cooling device 30 and the holder 100 is along the longitudinal axis down the endorectal cooling device 30 and the arm 110 of the holder 100. However, in other examples, the base 102 can have an oval shape, a circular shape, a triangular shape, or any other suitable shape.

The base 102 can be coupled with the arm 110. In at least one example, the arm 110 can have a length between about 15 centimeters and about 26 centimeters. Alternately, the length of the arm 110 can be between about 18 centimeters and about 23 centimeters, alternately between about 20 centimeters and about 22 centimeters, alternately about 21 centimeters. In at least one example, the arm 110 can have a length between about 5 centimeters and about 10 centimeters. Alternately, the length of the arm 110 can be between about 6 centimeters and about 9 centimeters, alternately between about 7 centimeters and about 8 centimeters, alternately about 7.6 centimeters. The width of arm 110 can be between about 3 centimeters and about 9 centimeters. Alternately, the width of the arm 110 can be between about 5 centimeters and about 7 centimeters, alternately about 6 centimeters. The arm 110 needs a length long enough so that the endorectal cooling device 30 can be inserted into the patient's rectum, and the arm 110 needs to have a width wide enough so that the arm 110 does not twist and has sufficient strength to hold the endorectal cooling device 30 in place.

To achieve the desired stability and strength while minimizing weight and/or cost of manufacturing the holder 100, as illustrated, the arm 110 can include two longitudinal beams 111, 112 extending along a longitudinal axis of the arm 110 and at least one cross-beam 113 connecting the two longitudinal beams. In at least one example, the two longitudinal beams 111, 112 can be substantially parallel to one another. In some examples, the two longitudinal beams 111, 112 can be at an angle in relation to one another. In at least one example, as illustrated in FIGS. 2A-2C, the arm 110 can include one cross-beam 113. In some examples, as illustrated in FIGS. 4A-4B, the arm 110 can include two cross-beams to further provide rigidity in the arm 110 and/or prevent rotation or twisting of the arm 110. In other examples, more than two cross-beams 113 can be included without deviating from the scope of the present disclosure.

In at least one example, to allow for adjustment of the holder 100 to accommodate different patients, the arm 110 can be rotatable in relation to the base 102. In some examples, the arm 110 can be immobile in relation to the base 102. In some examples, the arm 110 may be rotatable in relation to the base 102 but can be subject to sufficient friction or stopping force to prevent undesired movement unless a threshold force is enacted upon the arm 110. In some examples, the arm 110 may be rotatable in relation to the base 102 and be locked in the desired position.

In at least one example, as illustrated herein, the arm 110 may be coupled with the base 102 with a pin 150. The pin 150 can include a head 152 and a shaft 154 extending from the head 152. The base 102 can include one or more projections 106. As shown in FIG. 2C, the projections 106 form holes 1060, and the longitudinal beams 111, 112 form holes 1110, 1120. The holes 1060 of the projections 106 can be aligned with the holes 1110, 1120 of the arm 110. When the arm 110 is aligned with the projections 106, the shaft 154 of the pin 150 can be inserted through the holes 1060 of the projections 106 and the holes 1110, 1120 of the arm 110. The head 152 of the pin 150 prevents the pin 150 from sliding all the way through the arm 110 and the projections 106. In at least one example, as illustrated in FIGS. 2A-2C, the head 152 of the pin 150 can have substantially a rectangular shape. In some examples, as illustrated in FIGS. 4A-5D where the arm 110 is translatable along the base 102, the head 152 of the pin 150 can have substantially a circular shape to prevent collision with the base 102. In some examples, a cap (not shown) can be coupled to the shaft 154 of the pin 150 opposite the head 152 to secure the pin 150 in place.

As illustrated in FIGS. 2A-2C, the projections 106 can be substantially rectangular, but the projections 106 can be any suitable shape without deviating from the scope of the disclosure. In at least one example, the base 102 includes one projection 106, and the longitudinal beams 111, 112 of the arm 110 are positioned on either side of the projection 106. In at least one example, the base 102 can include a plurality of projections 106 which form gaps 107 in between the projections 106. The gaps 107 are operable to receive the arm 110. As illustrated herein, the base 102 includes three projections 106 to form two gaps 107 such that the two longitudinal beams 111, 112 can be received in the two gaps 107.

In at least one example, the arm 110 can be coupled to the base 110 proximate the rear end 104 of the base 102. As illustrated in FIGS. 2A-2C, the holder 100 may be configured such that the arm 110 extends from the rear end 104 away from a front end 103 of the base 102, the front end 103 being opposite the rear end 104. In some examples, as illustrated in FIGS. 1A-1C, the holder 100 may be configured such that the arm 110 extends from the rear end 104 towards the front end 103 of the base 102. In some examples, the arm 110 can be rotated about the base 102 to move between the two configurations as needed. Either of the two configurations may be used depending on how far away the patient is from the base 102 of the holder 100.

The receiver 120 is coupled with the arm 110 opposite the base 102. As shown in FIGS. 1A-1C, the receiver 102 is operable to receive at least a portion of the endorectal cooling device 30 and maintain the position of the endorectal cooling device 30. The receiver 102 has a frame 121 which forms a receiving aperture 122 which is operable to receive the endorectal cooling device 30.

In at least one example, the receiver 120 is pivotably coupled with the arm 110. The arm 110 can include a bracket 114 operable to receive the receiver 120. The bracket 114 can include a bottom portion 115 and two branches 116, 117 extending from opposing ends of the bottom portion 115. The bottom portion 115 can be coupled to and/or extending from the longitudinal beams 111, 112. The bottom portion 115 can extend substantially perpendicular to the longitudinal beams 111, 112, and the two branches 116, 117 can extend substantially perpendicular to the bottom portion 115. As can be seen in FIG. 2C, the branch 116 can include a protrusion 1160 therefrom, and the branch 117 can include a protrusion 1170 extending therefrom. In at least one example, as illustrated herein, the protrusion 1160 can extend from the branch 116 towards the branch 117, and the protrusion 1170 can extend from the branch 117 towards the branch 116. The receiver 120 includes two holes or recesses 1210 formed in opposing sides of the frame 121 corresponding to the positions of the protrusions 1160, 1170. The protrusions 1160, 1170 can be inserted into the corresponding hole or recess 1210. Accordingly, the receiver 120 is received in the bracket 114 of the arm 110 and can be pivoted about the protrusions 1160, 1170. As the receiver 120 is pivotably coupled with the arm 110, the receiver 102 and the endorectal cooling device 30 can be pivoted to ensure the proper orientation and positioning of the endorectal cooling device 30.

As mentioned above, the receiver 120 can have a frame 121 which forms a receiving aperture 122. The receiving aperture 122 is shaped to receive the endorectal cooling device 30. For example, the receiving aperture 122 can be substantially rectangular in shape. In some examples, the receiving aperture 122 can have a width between about 39 millimeters and 42 millimeters and a height between about 34 millimeters and about 36 millimeters. In some examples, the width of the receiving aperture 122 can be between about 40 millimeters and 42 millimeters, alternately between about 41 millimeters and 42 millimeters, alternately about 41.1 millimeters. In some examples, the height of the receiving aperture 122 can be between about 31 millimeters and about 34 millimeters, alternately between about 31 millimeters and about 32 millimeters, alternately about 32.25 millimeters. Accordingly, the receiving aperture 122 is operable to securely receive the endorectal cooling device 30 via friction fit to maintain the position of the endorectal cooling device 30. It is contemplated that the size and shape of the receiving aperture 122 can be different, so long as the receiving aperture 122 can securely receive the endorectal cooling device 30 without undesired movement of the endorectal cooling device 30. In some examples, coupling mechanisms may be included to couple the endorectal cooling device 30 with the receiver 120 within the receiving aperture 122. For example, adhesive may be included on the inner surfaces of the receiving aperture 122 that are operable to couple with the endorectal cooling device 30. In some examples, extensions may protrude outward from the receiving aperture 122 to abut against the endorectal cooling device 30.

As illustrated in FIGS. 3A and 3B, the receiver 120 may include further components to secure the position of the endorectal cooling device 30 within the receiving aperture 122 and to maintain the orientation of the endorectal cooling device 30 by preventing rotation of the endorectal cooling device 30. The endorectal cooling device 30 may include a ridge 36 that extends from the handle portion 34 of the endorectal cooling device 30. This ridge 36 are included in conventional endorectal cooling devices 30. Correspondingly, the receiving aperture 122 can be retrofit to the endorectal cooling device 30 and form a notch 124 operable to receive the ridge 36 on the endorectal cooling device 30. The ridge 36 being received in the notch 124 prevents the endorectal cooling device 30 from rotating. Accordingly, the positioning and orientation of the endorectal cooling device 30 is maintained.

As the receiver 120 can pivot in relation to the arm 110 and the arm 110 can rotate in relation to the base 102, the endorectal cooling device 30 can be positioned to be parallel to the table that the patient is on and is securely held in place during the procedure.

In at least one example, the receiver 120 can be removable from the arm 110. In some examples, the receiver 120 may be disposable and replaced for each procedure.

FIGS. 4A-5D illustrate alternative bases 202, 302 to increase maneuverability of the holder 100 to better position the endorectal cooling device 30 as each patient may require a different position. The bases 202, 302 as illustrated in FIGS. 4A-5D provide different configurations to provide a base 202, 302 so that the arm 110 can be translatable along a longitudinal axis of the base 202, 302. This can assist in adjusting the positioning of the endorectal cooling device 30 for the patient without having to lift, move, and place the base 102 of the holder 100 until the distance between the holder 100 and the patient is as desired. As illustrated in FIGS. 4A-5D, each of the different bases 202, 302 are configured such that the arm 110 and the receiver 120 as discussed above can fit with each base 202, 302. Accordingly, as discussed herein, the bases 102, 202, 302 discussed herein are interchangeable, so the bases 102, 202, 302 can be swapped out as needed depending on the patient. To change the base 102, 202, 302, the pin 150 can be removed, the arm 110 can then be separated from the protrusions 106, and a new base 102, 202, 302 can be put into place and coupled to the arm 110 via the pin 150. Other mechanisms translate the arm 110 along the base 202, 302 can be contemplated without deviating from the scope of the disclosure.

FIGS. 4A-4B illustrate a holder 100 with a hydraulic base 202. The hydraulic base 202 includes a support 204 and a pedestal 206. The support 204 and the pedestal 206 are coupled to one another such that the pedestal 206 can translate along a longitudinal axis in relation to the support 204. The hydraulic base 202 can include a pump 212 and a piston 210 that are operable to move the pedestal 206 in relation to the support 204. As the pump 212 either pushes fluid into the piston 210 or pulls fluid from the piston 210, the piston 210 translates, causing either the support 204 or the pedestal 206 to move in relation to one another. In the example illustrated in FIGS. 4A-4B, the pump 212 can be hydraulically coupled to the piston 210 via a hydraulic coupler 214. In at least one example, the hydraulic coupler 214 can include one or more valves. In some examples, the hydraulic coupler 214 can include one or more tubing to provide some separation from the components of the holder 100. In at least one example, as illustrated in FIGS. 4A-4B, the pump 212 and/or the piston 210 can include a syringe.

In some examples, as illustrated in FIGS. 4A and 4B, as the piston 210 moves, the pedestal 206 translates back and forth along the longitudinal axis of the hydraulic base 202 by sliding away from and towards the support 204. As the arm 110 is coupled to the hydraulic base 202 via the protrusions 106 on the pedestal 206, the arm 110 translates along the longitudinal axis along with the pedestal 206. In some examples, the arm 110 can be coupled to the hydraulic base 202 via the support 204, and the support 204 may translate instead of the pedestal 206.

In at least one example, as illustrated in FIGS. 4A and 4B, the hydraulic base 202 can include one or more guides 208. In some examples, the guides 208 can be coupled to and/or extend from the support 204, and the pedestal 206 is movably coupled or seated on the guides 208. For example, the guides 208 can serve as railing along which the pedestal 206 slides or translates along the longitudinal axis. In some examples, the guides 208 can be coupled to and/or extend from the pedestal 206, and the guides are received in the support 204. For example, the support 204 may remain in place and includes channels to receive the guides 208. As the pedestal 206 translates along the longitudinal axis due to the movement of the piston 210, the guides 208 remain within the channels in the support 204 but translate with the pedestal 206. The guides 208 can maintain aligned connection between the support 204 and the pedestal 206. For example, the guides 208 prevent lateral rotation of the pedestal 206 and subsequently the arm 110.

FIGS. 5A-5D illustrate a holder 100 with a rack base 302. The rack base 302 can include a support 304 and one or more guides 308 that is movably coupled to a cage 320. For example, the guides 308 can serve as railing along which the cage 320 slides or translates along the longitudinal axis. The cage 320 can include the protrusions 106 which are used to couple to the arm 110. The support 304 can include a guide rack 306 which can include a plurality of crests and valleys. The rack base 302 can include a knob 324 which, when turned, is operable to move the cage 320 along the guides 308. For example, when the knob 324 is turned, a cage rack 322 is turned. As the cage rack 322 turns, the crests on the cage rack 324 are moved into a corresponding valley on the guide rack 306, and the crests on the guide rack 306 are moved into a corresponding valley on the cage rack 322. This moves the cage rack 322 and the cage 320 longitudinally along the guide rack 306 and the support 304.

In at least one example, as illustrated in FIG. 5C, after the position of the cage 320 and correspondingly the arm 110 of the holder 100 is set, force 110F of the arm 110 is enacted on the cage 320. The force 110F can be imparted on the holder 100 by the endorectal cooling device 30 in the patient. As the projections 106 are disposed proximate the rear of the cage 320, the force 110F places torque 320T on the cage 320 and prevents the cage 320 from undesired movement backwards. In at least one example, as illustrated in FIG. 5D, a wedge 400 can be placed on the guide rack 306 to mechanically prevent the cage 320 from undesired movement. For example, to prevent backward movement, a wedge 400 can be placed behind the cage 320. To prevent forward movement, a wedge 400 can be placed in front of the cage 320.

FIGS. 6A-7C illustrate a holder 100 with a base 602 with alternative features that create a secure coupling with the surface 14, 20. As illustrated in FIGS. 6A-7C, the receiver 120, the arm 110, and the coupling between the arm 110 and the base 602 can be the same or similar to any combination of the features discussed above. With the base 602 as illustrated in FIGS. 6A-7C, the base 602 and the surface 20 of the positioning system 16 is coupled without modifying the positioning system 16. Accordingly, the base 602 is retrofit to work with the positioning system 16 without any modifications needed to the positioning system 16.

As illustrated in FIGS. 6A and 6B, the base 602 includes a top plate 620 and a bottom plate 610. The bottom plate 610 is operable to couple with the top plate 620 with the system base 20 positioned between the top plate 620 and the bottom plate 610. Accordingly, when the bottom plate 610 is coupled with the top plate 620, the base 602 is coupled with the system base 20 to provide a secure foundation for the holder 100. The bottom plate 610 can include one or more fasteners 612, 614 operable couple the top plate 620 with the bottom plate 610. The fasteners 612, 614 can be operable to be received by the top plate 620 to couple the bottom plate 610 with the top plate 620. In at least one example, the top plate 620 can include one or more apertures 622, 624 operable to correspondingly receive the fasteners 612, 614. For example, as illustrated in FIGS. 6A and 6B, the fasteners 612, 614 can include a screw 612. The screw 612 can be operable to be inserted into and through a corresponding aperture 622 formed in the top plate 620. The base 602 can include a nut 630 operable to be threadedly coupled with the screw 612 through extending through the top plate 620 to couple the top plate 620 with the bottom plate 610. As illustrated in FIG. 6B, the nut 630 includes an aperture 632 with threads 634 that correspond with the screw 612. Accordingly, when the screw 612 is received in the aperture 632, the threads 634 securely receive the screw 612 to provide a secure coupling. As the nut 630 is tightened on the screw 612, the top plate 620 is brought closer to the bottom plate 610. In at least one example, the fasteners 612, 614 can include a post 614 that is operable to extend from the bottom plate 610 and be received in a corresponding aperture 624 in the top plate 620. The post 614 can help prevent rotation or undesired movement between the top plate 620 and the bottom plate 610.

While FIGS. 6A and 6B illustrate that the fasteners 612, 614 extend from the bottom plate 610 to be received by the top plate 620, in some examples, any of the fasteners 612, 614 can extend from the top plate 620 to be received by the bottom plate 610 without deviating from the scope of the present disclosure. Also, other forms of coupling the top plate 620 with the bottom plate 610 can be utilized without deviating from the scope of the disclosure. While FIGS. 6A-7C illustrate two fasteners 612, 614, the number and type of fasteners 612, 614 can be interchanged without deviating from the scope of the present disclosure. The combination of fasteners 612, 614 discussed herein help prevent rotation and translation of the top plate 620 in relation to the bottom plate 610 while providing sufficient coupling to prevent undesired movement in relation to the system base 20.

In at least one example, the top plate 620 and/or the bottom plate 610 can include an adhesive which permits the base 602 to be removably coupled to the system base 20 and/or the surface 14. The adhesive may include a micro-suction tape. In some examples, the base 602 does not include adhesives.

FIGS. 6A-7C illustrate the nut 630 having a triangular shape. In other examples, the nut 630 can have other shapes such as circular, rectangular, ovoid, or any other suitable shape without deviating from the scope of the present disclosure.

FIGS. 7A-7C illustrate the base 602 being coupled with the system base 20. The bottom plate 610 is operable to be positioned against a bottom surface 24 of the system base 20 while the top plate 620 is operable to be positioned against a top surface 22 of the system base 20. The top surface 22 is opposite the bottom surface 24.

As illustrated in FIG. 7B, the bottom surface 24 of the system base 20 for the positioning system 16 forms a recess 26. Also, as illustrated in FIGS. 7B and 7C, the bottom surface 24 of the system 20 forms a channel 28. The bottom plate 610 is operable to be received in the recess 26 formed in the bottom surface 24 of the system base 20, and the top plate 620 is operable to be positioned on the top surface 22 of the system base 20 opposite the bottom surface 24. Accordingly, the system base 20 is sandwiched between the top plate 620 and the bottom plate 610. As the bottom plate 610 is received in the recess 24, the bottom plate 610 does not create stability issues for the positioning system 16.

To couple the top plate 620 with the bottom plate 610, the fastener(s) 612, 614 are operable to be inserted through the channel 28 in the system base 20 and through the corresponding aperture(s) 622, 624 formed in the top plate 620.

The recess 26 and channel 28 are utilized in the positioning system 16 to move the leg support 17 where the patient can rest their legs. The recess 26 and the channel 28 are formed in the system base 20 for conventional positioning systems 16. Accordingly, the base 602 is retrofit to the system base 20 so that the base 602 can be retrofittedly coupled with system base 20 without requiring any modifications to the system base 20 and/or the positioning system 16.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

What is claimed is:

1. An endorectal cooling device holding comprising:
a base operable to be coupled to a surface;
an arm extending from the base; and
a receiver pivotably coupled with the arm, the receiver forming a receiving aperture operable to receive an endorectal cooling device and maintain the position of the endorectal cooling device.

2. The holder of claim 1, wherein the receiving aperture is substantially rectangular.

3. The holder of claim 1, wherein the receiving aperture has a width between about 39 millimeters and 42 millimeters and a height between about 31 millimeters and about 36 millimeters.

4. The holder of claim 1, wherein the receiving aperture forms a notch operable to receive a ridge on the endorectal cooling device.

5. The holder of claim 1, wherein the base includes an adhesive which permits the base to be removably coupled to the surface.

6. The holder of claim 1, wherein the base includes a top plate and a bottom plate, wherein the bottom plate is operable to be received in a recess formed in a bottom surface of a system base for a positioning system, wherein the top plate is operable to be positioned on a top surface of the system base opposite the bottom surface.

7. The holder of claim 6, wherein the bottom plate includes a fastener that is operable to be received by the top plate, wherein the fastener is operable to couple the top plate with the bottom plate.

8. The holder of claim 7, wherein the fastener is operable to be inserted through a channel in the system base and through an aperture formed in the top plate.

9. The holder of claim 7, wherein the fastener includes a screw, wherein the base includes a nut operable to be threadedly coupled with the screw extending through the top plate to couple the top plate with the bottom plate.

10. The holder of claim 1, wherein the arm is rotatable in relation to the base.

11. The holder of claim 1, wherein the arm is translatable along a longitudinal axis of the base.

12. The holder of claim 1, wherein the receiver is pivotable in relation to the arm.

13. A system comprising:
an endorectal cooling device; and
a holder operable to receive and maintain the position of the endorectal cooling device, the holder including:
a base operable to be coupled to a surface;
an arm extending from the base; and
a receiver pivotably coupled with the arm, the receiver forming a receiving aperture operable to receive the endorectal cooling device.

14. The system of claim 13, wherein the receiving aperture is substantially rectangular.

15. The system of claim 13, wherein the receiving aperture has a width between about 39 millimeters and 42 millimeters and a height between about 31 millimeters and about 36 millimeters.

16. The system of claim 13, wherein the receiving aperture forms a notch operable to receive a ridge on the endorectal cooling device.

17. The system of claim 13, wherein the base includes an adhesive which permits the base to be removably coupled to the surface.

18. The system of claim 13, wherein the base includes a top plate and a bottom plate, wherein the bottom plate is operable to be received in a recess formed in a bottom surface of a system base for a positioning system, wherein the top plate is operable to be positioned on a top surface of the system base opposite the bottom surface.

19. The system of claim 18, wherein the bottom plate includes a fastener that is operable to be received by the top plate, wherein the fastener is operable to couple the top plate with the bottom plate.

20. The system of claim 19, wherein the fastener is operable to be inserted through a channel in the system base and through an aperture formed in the top plate.

21. The system of claim 19, wherein the fastener includes a screw, wherein the base includes a nut operable to be threadedly coupled with the screw extending through the top plate to couple the top plate with the bottom plate.

22. The system of claim 13, wherein the arm is rotatable in relation to the base.

23. The system of claim 13, wherein the arm is translatable along a longitudinal axis of the base.

24. The system of claim 13, wherein the receiver is pivotable in relation to the arm.
